(19) United States
(12) Patent Application Publication
(10) Pub. No.: US 2026/0219221 A1
(43) Pub. Date: Jul. 30, 2026

(54) CONDUCTIVITY/RESISTIVITY CELL, WATER PURIFICATION SYSTEM, AND METHOD OF DETERMINING THE CONDUCTIVITY AND/OR RESISTIVITY OF A SAMPLE LIQUID

(71) Applicant: Merck Patent GmbH, Darmstadt (DE)

(72) Inventors: Pierre Caruso, Paris (FR); Laurent Moreau, Versailles (FR)

(21) Appl. No.: 19/143,315

(22) PCT Filed: Jan. 8, 2024
(86) PCT No.: PCT/EP2024/050296

§ 371 Date: Jun. 25, 2025

(30) Foreign Application Priority Data
Jan. 12, 2023 (EP) 22290070.6

(51) Int. Cl. G01N 27/07; G01N 27/08; G01N 33/18; G01R 27/22

(52) U.S. Cl. CPC G01N 27/07; G01N 27/08; G01N 33/1893; G01R 27/22

(57) ABSTRACT

The present application relates to a conductivity/resistivity cell, preferably for use in a water purification system, to a water purification system provided with the conductivity/resistivity cell, and to a method of determining the conductivity and/or resistivity of a sample liquid.

### TECHNICAL FIELD

The present application relates to a conductivity/resistivity cell, preferably for use in a water purification system, to a water purification system provided with the conductivity/resistivity cell, and to a method of determining the conductivity and/or resistivity of a sample liquid.

### BACKGROUND

Conductivity/resistivity is a widely used analytical parameter for water purity analysis, monitoring of reverse osmosis, cleaning procedures, control of chemical processes, and in industrial wastewater.

A conductivity sensor, also known as a conductivity probe or a conductivity electrode, is an analytical instrument that measures the ability of a solution to conduct an electrical current. It is the presence of ions in a solution that allow the solution to be conductive: the greater the concentration of ions, the greater the conductivity. In some applications the purity measurement is made as resistivity (the reciprocal of conductivity). The terms “conductivity sensor”, “conductivity probe” and “conductivity cell” are considered synonymous in the context of this disclosure, wherein the term “cell” is uniformly used for simplification.

A conductivity/resistivity cell in the context of this disclosure is a flow-through device for use in a water purification system. The conductivity/resistivity cell includes an arrangement of typically one or two pairs of “conductivity electrodes” placed in the flow passage through which the liquid to be tested flows so that the liquid to be tested contacts the electrodes, and the signal of which is evaluated by an electronic device (controller or meter) to determine or measure the conductivity/resistivity of the liquid to be tested.

The electrodes in the prior art and in the invention are typically made of platinum, gold-plated nickel, titanium, stainless steel, or graphite. The body or manifold of the conductivity/resistivity cell with the flow passage is generally made of a material that is electrically non-conductive. It can, for example, be made of glass or resin. Examples of suitable resins may, without limitation, be selected from the group consisting of epoxy resins, polyacetals, polyamides. A preferred polyacetal is polyoxymethylene (“POM”).

The main characteristic that differentiates types of conductivity/resistivity cells is the cell constant of the conductivity/resistivity cell, identified by the letter K. Electrode size, the distance between the electrodes, and the pattern of the electrical field present determine this cell constant. It is generally higher for cells with small electrodes that are spaced far apart, and it is generally lower for ones that have larger electrodes spaced closer together.

To measure the conductivity/resistivity, a transmitter and a controller for signal conditioning, as well as a connecting cable to the conductivity/resistivity cell are required. The system may further include a microprocessor to help automate the conductivity/resistivity measuring process.

Conductance is typically measured in milli Siemens (mS) or micro Siemens (μS). When using contacting conductivity electrodes, conductivity cell geometry affects the conductivity reading. In order to ensure standardization of electrical conductivity measurements, units of specific conductivity are used. Specific conductivity is expressed as milliSiemens per centimeter (mS/cm) or microSiemens per centimeter (μS/cm). Resistivity, which is the reciprocal of conductivity, is typically measured in ohm-meter (Q·m). Specific conductivity=measured conductivity (G)*cell constant (K).

A cell constant of 1.0 will produce a measured conductivity (G) approximately equal to the specific conductivity of a solution. However, a cell constant of 1.0 is not always an appropriate choice. For example, in solutions with very low conductivity, like pure or ultra-pure water, measuring surfaces of the electrodes must be placed closer together in order to produce a signal having sufficient intensity for the controller or conductivity meter. When the path length between conducting plates is reduced, the cell constant is also reduced to 0.1 or even 0.01. Conversely, when measuring high conductivity solutions, a longer path length (higher cell constant) of 10 or 100 typically allows producing a more accurate reading.

Conductivity/resistivity measurement is a key parameter for a water purification system. It enables the control of the water purification processes and gives insights to the end user on the water quality delivered by the water purification product.

Depending on the water quality to be measured the appropriate cell coefficient (K) and the electronic hardware shall be defined and fine-tuned.

On a typical water purification system, the typical range of water quality to be measured is from 5000 μS/cm (concentrated tap feed water) to 80 Megaohms·cm (cold ultra-pure water). As a consequence, there is a huge range, i.e. a 400,000 fold ratio, on the water impedance to be measured by the electrical circuit of the electronic hardware.

To make it possible and guarantee an acceptable error on this measurement, this ratio is reduced with the cell coefficient. Typically, cell coefficients close to 1.0 for high conductivity, and close to 0.01 for high resistivity (low conductivity) are used. The ratio on the impedance to be measured in Ohms is reduced from 400,000 to 4,000.

The cell coefficient is a computation of the electrode's geometrical parameters (diameter, length, gap, orientation etc.). Cell coefficients close to 1.0 are often in parallel electrodes style, close from 0.01 often in concentric electrodes style. It would be very difficult and essentially impossible to produce a cell with coefficient K=0.01 with parallel electrodes or a cell with coefficient K=1.0 with concentric electrodes.

Depending on the water quality to be measured on the system (from tap water to ultra-pure water) and the desired flow rates, cells embedded in the system are a combination of parallel and concentric style with dedicated electronic hardware. From an industrial standpoint this is not preferred as cell geometry, electronic hardware cannot be mutualized and commonly used in respective devices due to small quantities of raw material, multiple assembling set-ups, multiple acquisition chains to be developed. Furthermore, concentric cells may lead to a too high pressure drop, particularly at higher flow rates.

A further drawback is the sensitivity of the cell coefficient to geometry of the variation of the mechanical part, especially for concentric cells. A small variation due to the machining or assembling process has a large impact on the cell coefficient and thus on the final measurement result.

Therefore, the cell coefficient cannot be determined by the estimated or rated dimension of the cell element but must be calibrated to define its exact cell coefficient. This calibration is costly as it is commonly performed on a calibration test bench with controlled water parameters.

An object to be solved is to provide a conductivity/resistivity cell, preferably for use in a water purification system for measuring the conductivity/resistivity of a sample liquid, preferably of pure or ultra-pure water, that can be used regardless the water quality and the cell coefficient of which can be determined without water calibration. Preferably, such conductivity/resistivity cell also needs to be produced in a simple process and in a cost efficient manner and/or must be reliable during use. The invention is also to provide a water purification system that includes and benefits from the improved conductivity/resistivity cell, and a method of determining the conductivity and/or resistivity of a method of determining the conductivity and/or resistivity of a sample liquid in a flexible range.

### SUMMARY

In order to solve the problems described above, the present invention provides a conductivity/resistivity cell with the features of claim 1, a water purification system with the features of claim 11, and a method of determining the conductivity and/or resistivity of a sample liquid with the features of claim 12. Preferred embodiments of the conductivity/resistivity cell and of the method are defined in the respective dependent claims.

The invention in particular provides a conductivity/resistivity cell comprising a manifold including a flow path through which a sample liquid to be measured can pass, and a pair of cylindrical electrodes each having a longitudinal central axis, a diameter d and an axial end face, wherein the cylindrical electrodes are mounted in the manifold such that the axial end faces of the cylindrical electrodes face each other across a gap located in the flow path with a distance e and are preferably aligned with a longitudinal direction of the flow path.

Preferably, the cylindrical electrodes are mounted in the manifold such that their longitudinal central axes are parallel, preferably concentrical and aligned with each other and preferably perpendicular to the longitudinal direction of the flow path.

Preferably, the manifold is configured such that an axial position of at least one of the cylindrical electrodes, preferably of both, can be adjusted relative to the other to adjust the distance e between the axial end faces of the electrodes facing each other across the gap.

Preferably, the manifold includes a receptacle configured to respectively accommodate electrodes with different diameters d.

Preferably, the diameter d of the cylindrical electrodes is between 5 and 30 mm.

Preferably, the axial end faces of the cylindrical electrodes are flat and preferably perpendicular to the longitudinal central axes and more preferably aligned with the longitudinal direction of the flow path.

Preferably, the flow path is dimensioned such that a maximum flow rate of the sample liquid through the flow path is between 3 l/h and 600 l/h and a pressure drop is 500 mbar or less at the maximum flow rate.

Preferably, a cell coefficient K of the conductivity/resistivity cell is between 0.1 and 1.0 and is preferably adjustable.

Preferably, the cell coefficient K of the conductivity/resistivity cell is determined by the following formula:

$$K = e / {( π \times d / 2 )}^{2}$$

wherein e is the distance between the axial end faces of the electrodes across the gap, and d is the diameter of the electrodes.

Preferably, the diameter d of the cylindrical electrodes is between 9 and 13 mm, preferably between 10 and 12 mm, more preferably between 10.5 and 11.5 mm, most preferably about 11 mm, and the distance e between the axial end faces of the cylindrical electrodes facing each other across the gap is between about 1 mm for the cell coefficient K of 0.1 and about 10 mm for the cell coefficient K of 1.0.

The invention in particular also provides a water purification system comprising a conductivity/resistivity cell according to this invention.

The invention in particular further provides a method of determining the conductivity and/or resistivity of a sample liquid, the method comprising the steps of
(a) providing a conductivity/resistivity cell (1) according to the invention;
(b) setting the distance e between the axial end faces of the cylindrical electrodes facing each other across the gap located in and preferably aligned with a longitudinal direction of the flow path;
(c) determining the cell coefficient K by the following formula

$$K = e / {( π \times d / 2 )}^{2}$$

wherein e is the distance between the axial end faces of the electrodes across the gap, and d is the diameter of the electrodes, and
(d) determining the conductivity and/or resistivity of the sample liquid.

Preferably, the diameter d of the cylindrical electrodes is selected to be between 5 and 30 mm, preferably between 9 and 13 mm, preferably between 10 and 12 mm, more preferably between 10.5 and 11.5 mm, preferably about 11 mm, and the distance e between the axial end faces of the cylindrical electrodes facing each other across the gap is set to be between about 1 mm and about 10 mm.

Preferably, in the method of determining the conductivity and/or resistivity of a sample liquid steps (a) and (b) are performed during the assembly of the conductivity/resistivity cell.

### BRIEF DESCRIPTION OF THE DRAWINGS

FIG. 1 shows a schematic cross section of an exemplary conductivity/resistivity cell.

FIG. 2 shows a schematic perspective view of the exemplary conductivity/resistivity cell of FIG. 1.

### DETAILED DESCRIPTION

The conductivity/resistivity cell of the invention provides the advantages that the same probes/electrodes and the same manifold can be used regardless of the water quality to be measured. This will reduce cost on the raw material and on assembly as the volumes of identical components to be produced/purchased are higher.

The geometrical arrangement of the electrodes/probes will enable the cell coefficient determination without water calibration, only with easy dimension measurement. In some water purification systems, water calibration may not be mandatory for the end user but nevertheless has to be performed at least once to determine the cell coefficient. The invention reduces the cost of this operation in terms of process cost and capitalization (i.e., a test bench for water calibration is not required).

It is easy to adapt the cell coefficient for a next product generation, because only the gap size and the software parameters have to be adapted. The dimension of the gap is easily controlled via assembly process parameters that can be easily fine-tuned and confirmed.

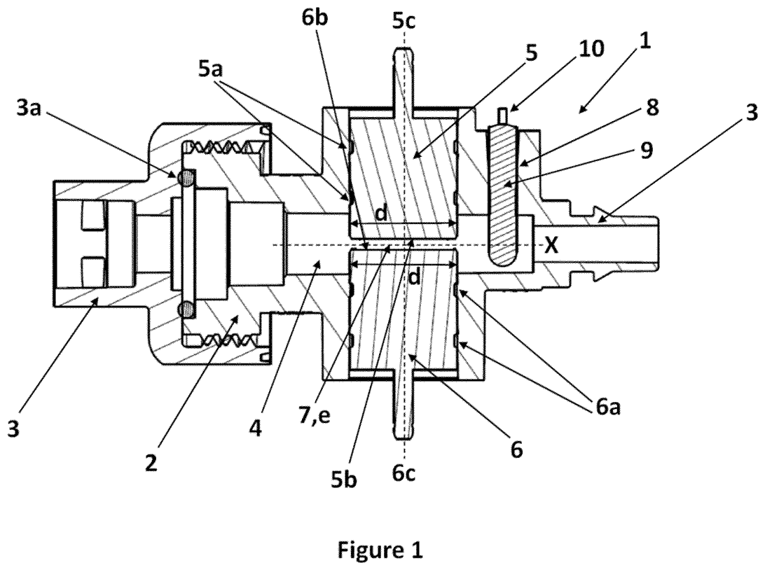

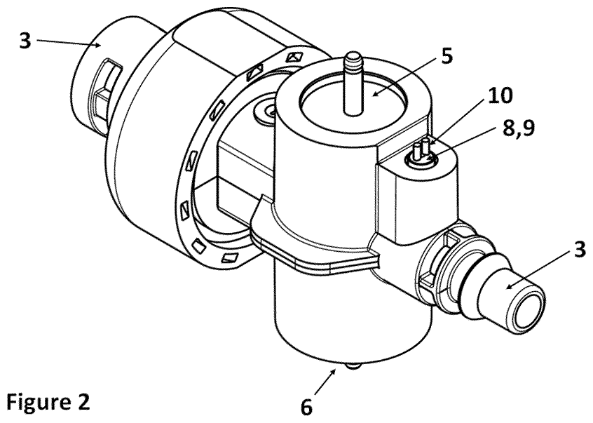

The invention is now described in detail on the basis of a preferred exemplary embodiment by reference to the attached exemplary schematic drawings, FIG. 1 and FIG. 2.

The conductivity/resistivity cell for a water purification system of the invention is now described in connection with an exemplary embodiment. It is noted that the specific design of the exemplary embodiment, in particular on the outside, is an example of a particular implementation but modifications are possible and are within the scope of the invention as long as the basic internal functionality is provided as defined by the claims.

The conductivity/resistivity cell 1 according to the exemplary embodiment comprises a body or manifold 2 including a flow path 4 through which a sample liquid to be measured can pass in use, and a pair of cylindrical electrodes 5,6 each having a longitudinal central axis 5c,6c, a diameter d and a preferably flat axial measuring end face 5b,6b.

The body or manifold 2 has a connector 3 at each of opposite ends for releasable connection to supply piping or hoses (not shown) for introducing/extracting the liquid to be tested into/out from the flow path 4. The connectors 3 may be threaded into or onto mating receptacles in/on the body or manifold 2 and sealed against the body or manifold 2 by means of a seal 3a, for example an O-ring or gasket (such threaded connection is shown as an example on the left side of the manifold). The connector or connectors may also be formed as an integral part of the body or manifold 2 as shown as an example on the right side of the manifold. In this case the number of parts and components is further reduced. The connectors 3 may have the same outer diameter but different inner diameters to accommodate pipes or hoses of different diameter/size depending on the rated flow rate of the system in which the cell is to be used.

The cylindrical electrodes 5,6 are mounted in the manifold 2 such that the axial end faces 5b,6b of the cylindrical electrodes 5,6 face each other with a distance e across a gap 7 located in the flow path 4, i.e., such that the planes of the flat end faces are parallel to each other. The flat axial measuring end faces 5b,6b are preferably perpendicular to the longitudinal axes 5c,6c and are more preferably aligned with a longitudinal direction X of the flow path 4. The direction of the distance e is thus perpendicular to the planes of the flat axial end faces 5b,6b and is preferably perpendicular to the direction of the flow through the section of the flow path 4, i.e. the longitudinal direction X of the flow path 4, where the end faces 5b,6b are located.

The cylindrical electrodes 5,6 are mounted in the manifold 2 such that their longitudinal central axes 5c,6c are parallel, preferably concentrical or more preferably aligned with each other and preferably perpendicular to the longitudinal direction X of the flow path 4 as shown in FIG. 1.

The manifold 2 is preferably configured such that an axial position of at least one of the cylindrical electrodes 5,6, preferably of both, can be adjusted relative to the other to adjust the distance e between the axial end faces 5b,6b of the electrodes 5,6 facing each other across the gap 7. Preferably, such adjustment of the distance e is done during the assembly of the present conductivity/resistivity cell. The distance e would then be left constant over the time of operation, for example, over the lifetime of the conductivity/resistivity cell. However, if the need arises, for example due to changing water purities and requirements, the distance e could also later (e.g. after assembly) be adapted to such changing conditions.

In order to be able to accommodate electrodes of different diameters in the same body or manifold 2, the manifold 2 may include a receptacle that is larger in diameter than the electrodes, and an insert or sleeve (not shown) that reduces the difference between the outer circumference of the electrodes and the inner circumference of the receptacle.

The electrodes are respectively sealed with respect to the flow path 4 by seals 5a,6a, for example in the form of O-rings or gaskets, or alternatively, by mechanical deformation of the body or manifold 2 through the introduction of the electrodes into the body or manifold 2. If a change of the axial position is not required after the initial mounting, the electrodes may be fluid-tightly sealed and fixed by any suitable mechanical fixation means or form-locking structure (for example, protrusions, lashes, or indentions) or by an adhesive or by mechanical deformation of the body or manifold 2 as described above.

In the exemplary embodiment of the conductivity/resistivity cell 1 shown in FIGS. 1 and 2 the body or manifold 2 is provided with a receptacle or bore 8 into which a thermistor 9 (i.e. a resistance thermometer) is inserted and fixed (for example by a suitable mechanical connection or by an adhesive) such that it can detect the temperature of the sample liquid to be measured passing in use through the flow path 4. The leading tip end with a measuring cell reaches into the flow path 4 and lead wires 10 of the thermistor 9 are accessible on the outside of the manifold 2. The receptacle or bore 8 into which the thermistor 9 is inserted is formed in the body or manifold 2 in the exemplary embodiment but may also be formed in one of the electrodes 5,6 (not shown) or in a separate section of the cell attached to the manifold 2 (not shown).

The cell coefficient K of the conductivity/resistivity cell 1 is determined by the following formula:

$$K = e / {( π \times d / 2 )}^{2}$$

wherein e is the distance between the axial end faces 5b,6b of the electrodes 5,6 across the gap 7, and d is the diameter of the electrodes 5,6.

With respect to the use of the conductivity/resistivity cell 1 in the water purification system a cell coefficient K of the conductivity/resistivity cell 1 is between 0.1 and 1.0 and is preferably adjustable by changing only the distance e between the axial end faces 5b,6b of the electrodes 5,6 across the gap 7.

Thus, the same electrodes and the same manifold can be used for a cell with a cell coefficient K between 0.1 and 1.0 in that the difference is made by the size of the gap e between the electrodes. That is, the diameter d of the electrodes 5,6 can be precisely measured prior to cell assembly, and the gap e may be measured after the assembly. Knowing the dimensions of the manifold in the section accommodating the electrodes it might be sufficient to measure the distance of the electrodes respectively protruding outside of the manifold to precisely determine their inside length and the size of the gap. Then, the cell coefficient K can be computed with these two measured values and does not necessarily need to be verified with water measurement in a calibration process.

This simple and precise determination of the cell coefficient cannot be performed with a concentric cell design because the gap between electrodes cannot be measured when assembled, and it is more difficult to perform on a parallel cell design as there are three parameters (diameter, length and distance between axis) to be measured and some are not directly measurable.

To reduce cost and size of the cell, the electrode diameter shall be as small as possible. With respect to the use of the conductivity/resistivity cell 1 in the water purification system, the diameter d of the cylindrical electrodes 5,6 is preferably between 5 and 30 mm.

In a preferred embodiment the combination of targeted cell coefficient and pressure drop limit leads to a preferred range of diameters d of between 9 and 13 mm, preferably between 10 and 12 mm, more preferably between 10.5 and 11.5 mm, and a preferred optimum diameter d of 11 mm and a distance e between the axial end faces 5b,6b of the cylindrical electrodes 5,6 facing each other across the gap 7 to be about 1 mm for the cell coefficient K of 0.1 and 10 mm for the cell coefficient K of 1.0. This optimum will be different in a product where flow rate would be different, maximal pressure drop would be different, and cell coefficient would be different.

With respect to the use of the conductivity/resistivity cell 1 in the water purification system, not only the sizes of the diameter d and of the gap e, but also the hydraulic parameters of the flow path shall be considered, especially the pressure drop of the liquid to be measured in the cell. Preferably, the flow path 4 is dimensioned such that a maximum flow rate of the sample liquid through the flow path 4 is between 3 l/h and 600 l/h and a pressure drop is 500 mbar or less at the maximum flow rate.

With respect to the electronic device (controller or meter) connected to the electrodes of the cell for evaluating their signal and determining or measuring the conductivity/resistivity of the liquid to be tested from tap to ultrapure water, the data acquisition needs to work with a wide range of equivalent resistors. It is common to use different gain resistors, each of them dedicated to a specific range. The cell is excited with an AC signal and the frequency of this signal is dependent of the cell and the impedance range to be measured (to accommodate the parasitic element). The filtering elements are chosen according to that frequency.

To address multiple cell coefficients, this approach is to be modified according to the invention. Multiple gain resistors are still used, but the frequency of the signal is dependent of the equivalent resistor to measure and is preferably automatically selected.

The filtering elements are selected to accommodate this frequency range (one gain resistor can be used at multiple frequencies) as is well known to the skilled person.

It is a new aspect to use the same electronic channel to measure the wide range of conductivity/resistivity with different cell coefficients depending on the water quality to be measured.

As the cell coefficient is known and recorded in the system memory, associated parameters such as frequency or readable range per gain can be different to accommodate the various cells coefficient. This data can be provided and selected by software settings. Using a dedicated electronic capable of reading high equivalent resistors permits to reduce the ratio required between cell coefficients.

1. A conductivity/resistivity cell (1) comprising:
a manifold (2) including a flow path (4) through which a sample liquid to be measured can pass, and
a pair of cylindrical electrodes (5,6) each having a longitudinal central axis (5c, 6c), a diameter d and an axial end face (5b, 6b),
wherein the cylindrical electrodes (5,6) are mounted in the manifold (2) such that the axial end faces (5b, 6b) of the cylindrical electrodes (5,6) face each other across a gap (7) located in the flow path (4) with a distance e and are optionally aligned with a longitudinal direction (X) of the flow path (4).

2. The conductivity/resistivity cell (1) according to claim 1, wherein the cylindrical electrodes (5,6) are mounted in the manifold (2) such that their longitudinal central axes (5c, 6c) are parallel, optionally concentrical and aligned with each other and optionally perpendicular to the longitudinal direction (X) of the flow path (4).

3. The conductivity/resistivity cell (1) according to claim 1, wherein the manifold (2) is configured such that an axial position of at least one of the cylindrical electrodes (5,6) can be adjusted relative to the other to adjust the distance e between the axial end faces (5b, 6b) of the electrodes (5,6) facing each other across the gap (7).

4. The conductivity/resistivity cell (1) according to claim 1, wherein the manifold (2) includes a receptacle configured to respectively accommodate electrodes (5,6) with different diameters d.

5. The conductivity/resistivity cell (1) according to claim 1, wherein the diameter d of the cylindrical electrodes (5,6) is between 5 and 30 mm.

6. The conductivity/resistivity cell (1) according to claim 1, wherein the axial end faces (5b, 6b) of the cylindrical electrodes (5,6) are flat and optionally perpendicular to the longitudinal central axes (5c, 6c) and more optionally aligned with the longitudinal direction (X) of the flow path (4).

7. The conductivity/resistivity cell (1) according to claim 1, wherein the flow path (4) is dimensioned such that a maximum flow rate of the sample liquid through the flow path (4) is between 3 l/h and 600 l/h and a pressure drop is 500 mbar or less at the maximum flow rate.

8. The conductivity/resistivity cell (1) according to claim 1, wherein a cell coefficient K of the conductivity/resistivity cell (1) is between 0.1 and 1.0 and is optionally adjustable.

9. The conductivity/resistivity cell (1) according to claim 8, wherein the cell coefficient K of the conductivity/resistivity cell (1) is determined by the following formula:
$$K = e / {( π \times d / 2 )}^{2}$$
wherein e is the distance between the axial end faces (5b, 6b) of the electrodes (5,6) across the gap (7), and d is the diameter of the electrodes (5,6).

10. The conductivity/resistivity cell (1) according to claim 9, the diameter d of the cylindrical electrodes (5,6) is between 9 and 13 mm, and the distance e between the axial end faces (5b, 6b) of the cylindrical electrodes (5,6) facing each other across the gap (7) is between about 1 mm for the cell coefficient K of 0.1 and about 10 mm for the cell coefficient K of 1.0.

11. A water purification system comprising a conductivity/resistivity cell (1) according to claim 1.

12. A method of determining the conductivity and/or resistivity of a sample liquid, the method comprising the steps of
(a) providing a conductivity/resistivity cell (1) according to claim 1;
(b) setting the distance e between the axial end faces (5b, 6b) of the cylindrical electrodes (5,6) facing each other across the gap (7) located in and optionally aligned with a longitudinal direction (X) of the flow path (4);
(c) determining the cell coefficient K by the following formula
$$K = e / {( π \times d / 2 )}^{2}$$
wherein e is the distance between the axial end faces (5b, 6b) of the electrodes (5,6) across the gap (7), and d is the diameter of the electrodes (5,6), and
(d) determining the conductivity and/or resistivity of the sample liquid.

13. The method of determining the conductivity and/or resistivity of a sample liquid according to claim 12, wherein the diameter d of the cylindrical electrodes (5,6) is selected to be between 5 and 30 mm, and the distance e between the axial end faces (5b, 6b) of the cylindrical electrodes (5,6) facing each other across the gap (7) is set to be between about 1 mm and about 10 mm.

14. The method of determining the conductivity and/or resistivity of a sample liquid according to claim 12, wherein steps (a) and (b) are performed during the assembly of the conductivity/resistivity cell.

15. The conductivity/resistivity cell (1) according to claim 1, wherein the manifold (2) is configured such that an axial position of both cylindrical electrodes (5,6) can be adjusted relative to the other to adjust the distance e between the axial end faces (5b, 6b) of the electrodes (5,6) facing each other across the gap (7).

16. The conductivity/resistivity cell (1) according to claim 9, wherein the diameter d of the cylindrical electrodes (5,6) is about 11 mm and the distance e between the axial end faces (5b, 6b) of the cylindrical electrodes (5,6) facing each other across the gap (7) is between about 1 mm for the cell coefficient K of 0.1 and about 10 mm for the cell coefficient K of 1.0.

17. The method of determining the conductivity and/or resistivity of a sample liquid according to claim 12, wherein the diameter d of the cylindrical electrodes (5,6) is selected to be between 9 and 13 mm and the distance e between the axial end faces (5b, 6b) of the cylindrical electrodes (5,6) facing each other across the gap (7) is set to be between about 1 mm and about 10 mm.

18. The method of determining the conductivity and/or resistivity of a sample liquid according to claim 12, wherein the diameter d of the cylindrical electrodes (5,6) is selected to be about 11 mm and the distance e between the axial end faces (5b, 6b) of the cylindrical electrodes (5,6) facing each other across the gap (7) is set to be between about 1 mm and about 10 mm.
